(19) United States
(12) Patent Application Publication
(10) Pub. No.: US 2025/0144365 A1
(43) Pub. Date: May 8, 2025

(54) STAGGERED PACKAGING TRAY ASSEMBLY FOR A MEDICAL DEVICE

(71) Applicant: BIOTRONIK AG, Buelach (CH)

(72) Inventors: Andreas Bodmer, Windisch (CH); Lucien Pistol, Rielasingen-Worblingen (DE); Marc Gianotti, Wiesendangen (CH); Wolfgang Bley, Basel (CH); Ulf Fritz, Bargen (CH)

(21) Appl. No.: 18/727,995

(22) PCT Filed: Jan. 19, 2023
(86) PCT No.: PCT/EP2023/051227

§ 371 Date: Jul. 10, 2024

(30) Foreign Application Priority Data
Feb. 9, 2022 (EP) 22155835.6

(51) Int. Cl. A61M 25/00

(52) U.S. Cl. CPC A61M 25/002

(57) ABSTRACT

A packaging for a medical device includes an outer packaging defining an internal space. A first and a second tray are configured to be arranged in the internal space. The second tray includes at least one protrusion on a bottom side of the second tray. The first tray includes at least one recess on a top side of the first tray in alignment with the protrusion. The second tray is configured to be arranged on the first tray so that the at least one protrusion engages with the at least one recess.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2023/051227, which was filed Jan. 19, 2023, which application claimed priority from EP Application Serial No. 22155835.6, which was filed Feb. 9, 2022.

### FIELD OF THE INVENTION

The present invention relates to a packaging for a medical device.

### BACKGROUND

A general challenge regarding packages for medical devices is to yield a reduced profile, volume and weight, while guaranteeing that the package can be handled in a convenient fashion by a user, particularly by a physician/clinician.

### SUMMARY OF THE INVENTION

A preferred packaging for a medical device includes an outer packaging defining an internal space. A first and a second tray are configured to be arranged in the internal space. The second tray includes at least one protrusion on a bottom side of the second tray. The first tray includes at least one recess on a top side of the first tray in alignment with the protrusion. The second tray is configured to be arranged on the first tray so that the at least one protrusion engages with the at least one recess.

### BRIEF DESCRIPTION OF THE DRAWINGS

In the following, embodiments of the present invention as well as further features and advantages of the present invention are described with reference to the Figures, wherein

FIG. 1 shows an exploded view of an embodiment of a packaging of the present invention for a medical device, particularly in form of a catheter system;

FIG. 2 shows a top view onto a first tray of the embodiment of the packaging shown in FIG. 1;

FIG. 3 shows a top view onto a second tray of the embodiment of the packaging shown in FIG. 1; and

FIG. 4 shows a top view onto the first and the second tray shown in FIGS. 1 to 3 being stacked on top of one another.

### DETAILED DESCRIPTION OF THE PREFERRED EMBODIMENTS

A preferred packaging for a medical devices includes an outer packaging defining an internal space of the outer packaging. At least a first and a second tray are configured to be arranged in the internal space of the outer packaging, wherein the second tray includes at least one protrusion on a bottoms side of the second tray, and wherein the first tray includes at least one recess on a top side of the first tray, the recess being aligned with the at least one protrusion, wherein the second tray is configured to be arranged on the first tray so that the protrusion engages with the recess.

The packaging is sterilization compatible. Due to the stacked trays, several commonly observed problems that conventional tray packaging systems exhibit are resolved. In particular, the staggered tray assembly exhibits a reduced profile, volume and weight.

Thereby, the staggered tray assembly is easier to transport and store. Accordingly, shelf space requirements at manufacturer, distributor, and hospital sites are reduced, and more inventory can be stored at any time.

According to a preferred embodiment of the packaging, the at least one protrusion includes a base portion forming a spacer and an engaging portion protruding from the base portion, the engaging portion configured to be inserted (particularly in a form fitting manner) into the at least one recess so that a face side of the base portion rests on the top side of the first tray. Particularly, the at least one protrusion and the at least one recess are deep drawn into the respective tray.

Further, in an embodiment, the at least one protrusion and the at least one recess are tapered so as to induce a press-fit when the at least one protrusion is inserted into the respective recess and/or wherein the at least one protrusion and the at least one recess are configured to induce a snap fit upon insertion of the at least one protrusion into the at least one recess.

Further, according to an embodiment of the packaging, the second tray includes a plurality of protrusions on the bottom side of the second tray, and wherein the first tray includes a corresponding plurality of recesses on a top side of the first tray, each recess configured to be aligned with one of the protrusions, wherein the second tray is configured to be arranged on the first tray so that each protrusion engages with a corresponding recess. Particularly, the respective portion and the respective recess can be designed as described above with respect to the at least one protrusion and associated recess. Particularly, the trays can include two pairs of a protrusion and associated recess being arranged diagonally with respect to one another, when the two trays are stacked on top of one another as described above.

According to yet another embodiment, the first and/or the second tray each includes at least one well for receiving at least one component of the medical device, respectively.

According to a further embodiment of the invention, the first and/or the second tray includes at least one holding element configured to hold the respective component in place, when the respective component is accommodated in the associated well.

Further, in an embodiment, the packaging includes an inner packaging in form of a sterile pouch, the sterile pouch being configured to hermetically enclose the trays when the trays are arranged in the internal space of the outer packaging and carry the components of the medical device. Particularly, the outer packaging can be a cardboard box.

Furthermore, according to an embodiment of the packaging, the first tray includes at least a first material, and wherein the second tray includes at least a second material, the first and the second material being different from one another, wherein particularly the first and the second material differ with respect to at least one property selected from the group included of: absorption coefficient with respect to light (the absorption coefficient determines how far into a material light of a particular wavelength can penetrate before it is absorbed), elastic modulus, flexural modulus, density, color.

Further, in an embodiment, the first tray is softer than the second tray to form a cushion for the second tray for shock resistance.

Further, according to an embodiment, the second tray includes a light absorbing dye and/or includes a light stopping material (such as an aluminum foil), such that a light sensitive component of the medical device arranged on the first tray can be protected from overexposure to UV radiation.

Furthermore, in an embodiment of the packaging, the first and/or the second tray include a heat insulating component and/or a heat insulating material (such as heat reflective foil), such that a heat sensitive component of the medical device present in a space formed between the first and second tray can be protected from overexposure to heat or can stably maintain an inner temperature relative to an external temperature (e.g., so as to prevent the build-up of heat or cold during storage and transport).

Further, according to yet another embodiment of the packaging, the first and/or the second tray include a permeation regulating component and/or a permeation regulating material (e.g. a material impermeable to gases and/or liquids) such that a permeation sensitive component of the medical device present in a space formed between the first and second tray can be protected from a gas and/or a liquid (such as e.g. water and/or oxygen). Alternatively, the first and/or the second tray can include a semipermeable material to permit accelerated transport of a gas, particularly of ethylene oxide vapor, across the first and second trays.

Furthermore, preferably, the trays include or are formed out of a plastic material such as: polyethylene, polypropylene, polyethyleneterephthalate, glycol-modified polyethyleneterephthalate, polycarbonates as well as copolymers of the aforementioned materials.

Furthermore, according to a preferred embodiment, the first and/or second tray is formed by a deep drawing process out of a plastic material.

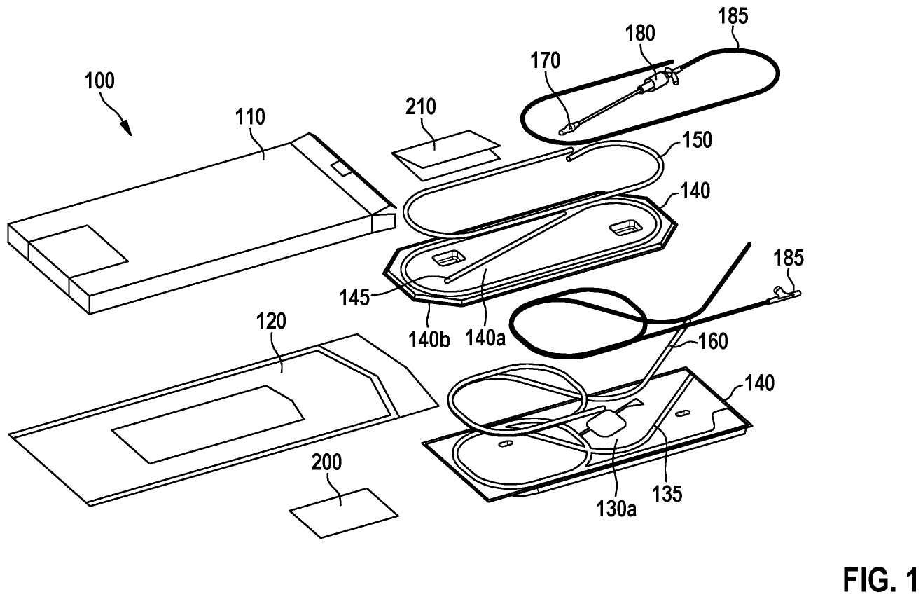

The present invention relates to a packaging 100 that includes at least a first and a second tray 130, 140 (or more than two such trays) being stacked on top of one another. Such a packaging is particularly suited to package medical devices including elongated components such as catheter systems. As shown in FIG. 1, besides the trays 130, 140, the packaging 100 can include an outer packaging 110, particularly shaped as a cardboard box, and an inner packaging 120, shaped e.g. as a sterile pouch for enclosing the first and second tray 130, 140. The packaging 100 can further include multiple transport coils 150 and 160, for protecting various assorted medical devices 170, 180, 185, 190 arranged on the trays 130, 140, a compliance card 200 and an IFU 210, as exemplary embodiments.

Particularly, in an embodiment, the medical device can be a catheter system, the catheter system including a support catheter 185, a handle 180 including a hemostatic valve, a dilatation catheter 170, and a balloon catheter 190. Furthermore, for protection, the support catheter 185 and dilatation catheter 170 can be protected by a transport coil 150 surrounding the latter components 170, 185 at least partially, whereas the balloon catheter 190 can be protected by transport coil 160 surrounding the balloon catheter 190 at least partially.

Each of the at least two trays 130, 140 can include a well 135, 145, wherein the balloon catheter 190 and the surrounding transport coil 160 are inserted in the well 135 of the first tray 130 while the support and dilatation catheter 170, 180, 185 and surrounding transport coil 150 are inserted in the well 145 of the second tray 140.

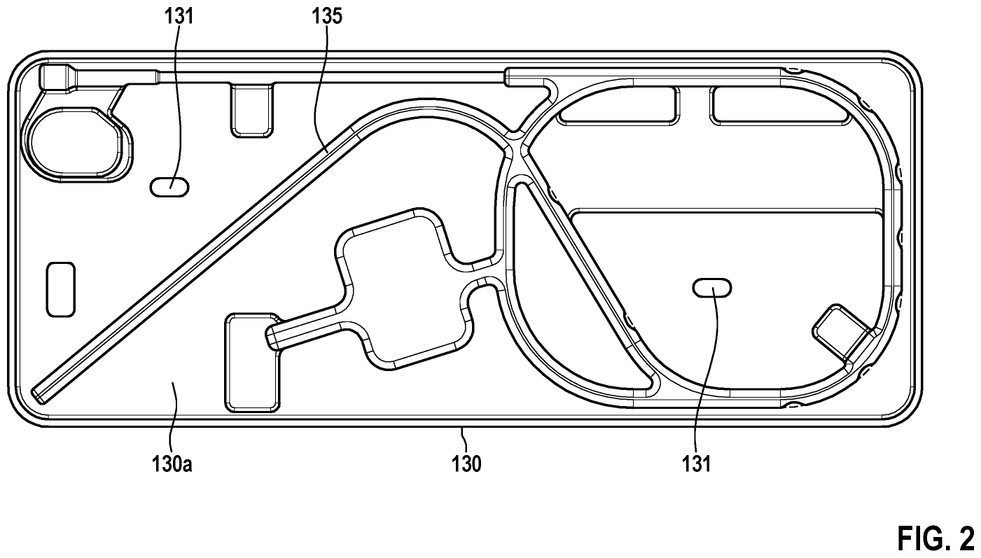

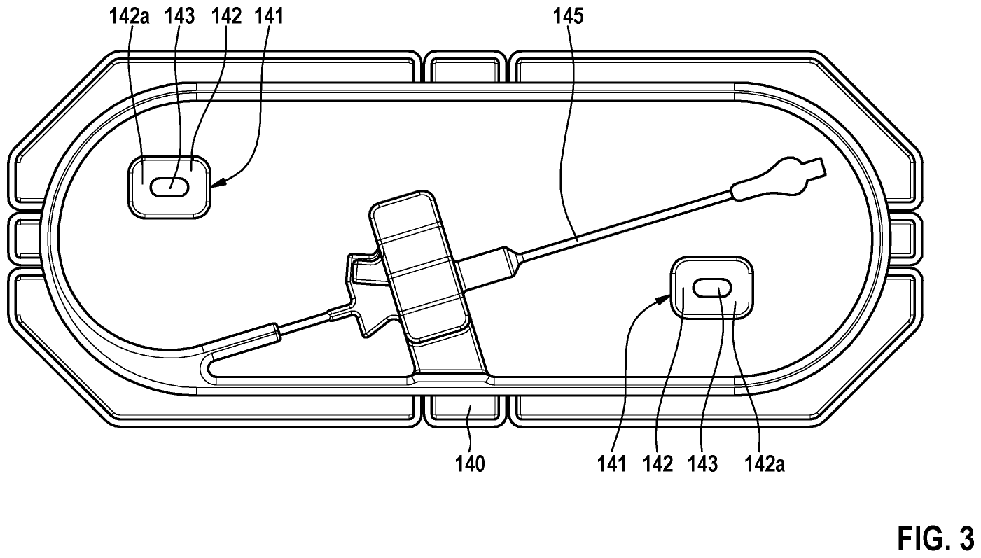

Furthermore, as shown in FIGS. 2 and 3, each tray 130, 140 preferably incorporates one or more press-fit recesses 131 and protrusions 141 that correspond in position, shape and height/depth with one another, so that two or more trays 130, 140 can be straightforwardly positioned on top and partially sunk into one another. Due to the overlapping depth, material or wall strength of trays 130, 140 can be reduced, and thus weight of the total package reduced. Particularly, the the respective protrusion 141 can include a base portion 142 forming a spacer and an engaging portion 143 protruding from the base portion/spacer 142, the engaging portion 143 being configured to engage, particularly in a form fitting manner, into the associated recess 131 so that a face side 142a of the base portion 142 rests on the top side 130a of the first tray 130.

Each tray 120, 130 can further contain elements such as holding elements, guiding inserts, or fixation embossments to temporarily lock said components of the medical device into the correspondingly shaped wells 135, 145, that in turn act as receptacles for the respective components, that are in turn placeable and lockable into each respective tray 130, 140. Each tray 130, 130 can be shaped as necessitated by the geometry of the underlying components/medical devices, thereby allowing the shapes of each tray 130, 140 to be different from one another and specifically adapted according to the packaging requirements.

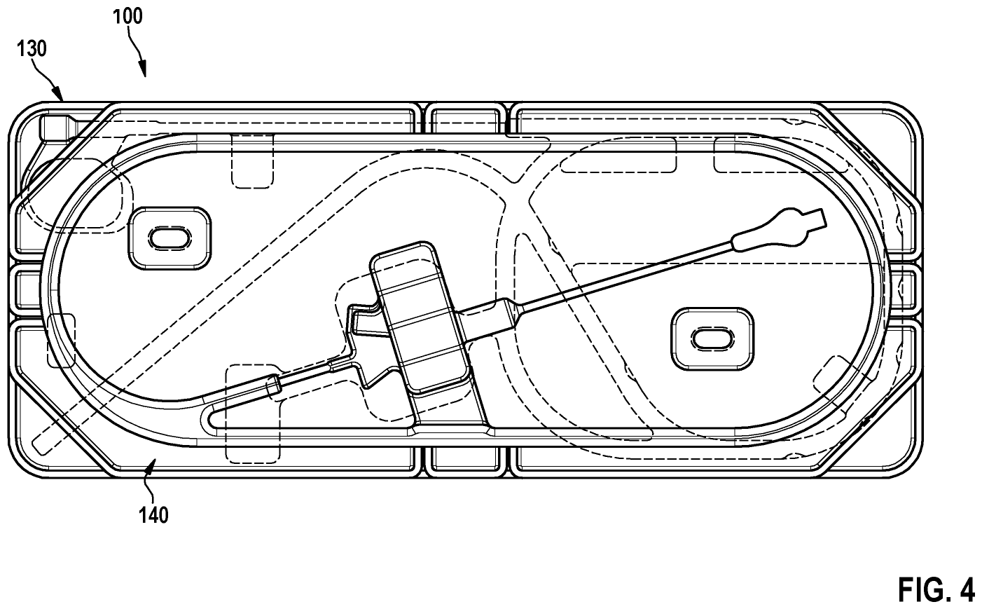

The protrusions 141 and recesses 131, or generally the corresponding embossments present on the two or more trays 130, 140 can be wedge-shaped or slightly tapered, or may contain horizontal protrusions, and corresponding cavities, such as to induce a press-fit, or snap fit assembly upon mutual contact. For separation of the locked trays 130, 140 (cf. FIG. 4), the user can overcome a tactile bump/resistance in order to release the locked trays 130, 140 from one another to access the contents of each respective tray 130, 140 at desired times.

The height/depth ratio of the protrusions/recesses 141, 131 (e.g. embossments) allows for adjustment of the residual height between the trays 130, 140 when locked to facilitate proper ventilation between the trays during ETO sterilization.

Due to the staggered arrangement, the packaging stability, crush and/or bending resistance of the trays 130, 140 is improved and inadverted movement of trays 130, 140 relative to one another during transport is avoided. The two or more trays 130, 140 can be packaged together as a single assembly in a sterile pouch 120, thereby additional packaging items such as blister foils for each tray 130, 140 can be avoided. Further, because the staggered assembly requires less height as compared to the mere stacking of two trays on top of each other, the overall packaging footprint can be considerably reduced.

Because the trays 130, 140 can be manufactured from a combination of dissimilar materials having different properties, certain properties, such as light absorption, elastic and flexural modulus, weight/density, color, among others, can be beneficially altered. Hence, the combination of the same, or dissimilar tray materials, can impart new desirable properties that are not available from single trays that are manufactured from the same material:

Improved shock resistance, provided by the addition of a first tray 130 formed from substantially flexible, soft, foamed, or generally cushioning materials to a second tray 140 formed from comparably more rigid, fragile and/or inflexible materials, and positioning the first tray 130 underneath the second tray 140. Thereby, fragile materials that might otherwise be prone to breaking when exposed to the rigors of transport, can be more safely protected.

Furthermore, by providing light absorbing dyes or light stopping materials, such as an aluminum foil, to the second tray 140, and positioning the second tray 140 on top of the first tray an improved light attenuation/absorption can be achieved, particularly such that light sensitive materials present on the first tray 130 can be protected from overexposure to elements, such as sun or UV-radiation.

Further, by providing heat insulating components and/or materials, such as a heat reflective foil, to the first and second tray, and positioning the second tray 140 on top of the first tray 130, improved insulating properties can be provided, particularly such that heat sensitive materials present in the space formed between the first and second tray 130, 140 can be protected from overexposure to heat. Alternatively, this tray configuration can be employed to stably maintain an inner temperature relative to an external temperature, to prevent the build-up of heat or cold during storage and transport.

Furthermore, an improved gas or liquid permeation and/or barrier properties can be achieved by providing permeation regulating components and/or materials, such as gas stop barriers, to the first tray 130 and/or to the second tray 140, and positioning the second tray 140 on top of the first tray 130, such that permeation sensitive materials present in the space formed between the first and second tray 130, 140 can be protected from water, and/or oxygen. Alternatively, semipermeable materials may be provided to permit accelerated transport of ethylene oxide vapor across the packaging barrier formed from the first and second trays 130, 140.

Preferably, the trays 130, 140 of the package 100 are preferably formed by a conventional deep drawing process. The staggered packaging assembly 100 according to the present invention is generally applicable to any product, including medical devices. Since the technology particularly utilizes conventional manufacturing processes, such as deep drawing, there are no fundamental technological challenges present.

1. A packaging for a medical device comprising:
an outer packaging defining an internal space; and
a first and a second tray configured to be arranged in the internal space, wherein the second tray comprises at least one protrusion on a bottom side of the second tray, and wherein the first tray comprises at least one recess on a top side of the first tray, the at least one recess being aligned with the at least one protrusion, wherein the second tray is configured to be arranged on the first tray so that the at least one protrusion engages with the at least one recess.

2. The packaging according to claim 1, wherein the at least one protrusion comprises a base portion forming a spacer and an engaging portion protruding from the base portion, the engaging portion being configured to be inserted into the at least one recess so that a face side of the base portion rests on the top side of the first tray.

3. The packaging according to claim 1, wherein the at least one protrusion and the at least one recess are deep drawn into the respective tray.

4. The packaging according to claim 1, wherein the at least one protrusion and the at least one recess are tapered so as to induce a press-fit when the at least one protrusion is inserted into the at least one recess and/or wherein the at least one protrusion and the at least one recess are configured to induce a snap fit upon insertion of the at least one protrusion into the at least one recess.

5. The packaging according to claim 1, wherein the second tray comprises a plurality of protrusions on the bottoms side of the second tray, and wherein the first tray comprises a corresponding plurality of recesses on a top side of the first tray, each recess being aligned with one of the plurality of protrusions, wherein the second tray is configured to be arranged on the first tray so that each of the plurality of protrusions engages with a corresponding one of the plurality of recesses.

6. The packaging according to claim 1, wherein the first and/or the second tray comprises at least one well for receiving a component of the medical device.

7. The packaging according to claim 6, wherein the first and/or the second tray comprises at least one holding element configured to hold the respective component in place, when the respective component is accommodated in the associated at least one well.

8. The packaging according to claim 1, comprising an inner packaging in form of a sterile pouch, the sterile pouch being configured to hermetically enclose the first tray and the second tray when they are arranged in the internal space.

9. The packaging according to claim 1, wherein the outer packaging is a cardboard box.

10. The packaging according to claim 1, wherein the first tray comprises a first material, and wherein the second tray comprises a second material, the first and the second material having at least one different property from one another, wherein the at least one different property is selected from the group consisting of: absorption coefficient with respect to light, elastic modulus, flexural modulus, density, and color.

11. The packaging according to claim 1, wherein the first tray is softer than the second tray to form a cushion for the second tray thereby creating shock resistance.

12. The packaging according to claim 1, wherein the second tray comprises a light absorbing dye and/or comprises a light stopping material, such that a light sensitive component of the medical device arranged on the first tray can be protected from overexposure to UV radiation.

13. The packaging according to claim 1, wherein the first and/or the second tray comprises a heat insulating component and/or a heat insulating material, such that a heat sensitive component of the medical device present in a space formed between the first and second tray can be protected from overexposure to heat or can stably maintain an inner temperature relative to an external temperature.

14. The packaging according to claim 1, wherein the first and/or the second tray comprise a permeation regulating component and/or a permeation regulating material such that a permeation sensitive component of the medical device present in a space formed between the first and second tray can be protected from a gas and/or a liquid, or wherein the first and/or the second tray comprise a semipermeable material to permit accelerated transport of a gas, across the first and second trays.

15. The packaging according to claim 1, wherein the first and or second tray is formed by a deep drawing process.
